(19) United States
(12) Patent Application Publication
(10) Pub. No.: US 2005/0268364 A1
(43) Pub. Date: Dec. 8, 2005

(54) Sun hat with an enlarged visor

(76) Inventor: Bog Kyu Kim, Nam-Gu (KR)

Correspondence Address: JORDAN AND HAMBURG LLP, 122 EAST 42ND STREET, SUITE 4000, NEW YORK, NY, 10168, US

(21) Appl. No.: 11/112,458

(22) Filed: Apr. 22, 2005

(30) Foreign Application Priority Data
Apr. 22, 2004 (KR) 2004-27936

(51) Int. Cl. A61F 9/00

(52) U.S. Cl. 2/12

(57) ABSTRACT

Disclosed is a sun hat including ear-covering portions, which provides sun protection for the front of face and ears of a wearer so as to minimize the danger of a bum through direct sunlight, by integrally extending the ear-covering portions from a peripheral portion of a visor installed at an elastic hair band of the sun hat, and by setting a visor installation angle to a relatively small acute angle. The sun hat includes a circular hair band of which a rear side is open and a visor integrally formed with the hair band at a front portion thereof. Ear-covering portions rewardly extend from a peripheral portion of the visor so as to provide sun protection for a front portion and ears in a face of a wearer. An installation angle of the visor installed at the hair band is set to a relatively small acute angle of about 30 to 45 degrees.

### BACKGROUND OF THE INVENTION

1. Field of the Invention

The present invention relates to a sun hat having ear-covering portions, more particularly to a sun hat having ear-covering portions, which provides sun protection for the front of face and ears of a wearer so as to minimize the danger of a burn through direct sunlight, by integrally extending the ear-covering portions from a peripheral portion of a visor installed at an elastic hair band of the sun hat, and by setting a visor installation angle, which is defined between a tangential line of the visor and a normal line projecting from the center lower portion of the hair band, to a relatively small acute angle.

2. Description of the Related Art

As shown in FIGS. 1, 2 and 5A, a conventional sun hat 1 comprises a circular elastic hair band 2 having an open rear portion and a visor 3 installed at a front side of the hair band 2. The visor 3 comprises a semitransparent body on which a UV-protection composition is coated at a predetermined thickness.

Referring to FIG. 2, when a user puts on the conventional sun hat 1, the visor 3 of the sun hat 1 only cover eyes of the wearer but it cannot cover ears of the wearer. At this time, a visor installation angle, which is defined between a tangential line of the visor and a normal line projecting from the center lower portion of the hair band, makes a certain angle of 60 degrees or so.

Because the visor 3 of the conventional sun hat 1 cannot cover both ears and cheeks of the wearer, the ears and the cheeks are exposed to the direct sunlight and therefore they may be easily sunburned.

Since the visor installation angle makes a certain angle of 60 degrees or so, the face of the wearer is spaced from an inner peripheral portion of the visor 3 at a distance of about 10 cm. Accordingly, a portion to be shaded in the face of the wearer may be changed in accordance with the position of the sun around the earth. Furthermore, the surface area of the portion to be shaded in the face of the wearer gets smaller and smaller. Although the visor 3 is installed at the sun hat 1, a nose of the face may be exposed to the sunlight and thereby the front portion and the nose in the face of the wearer get sunburned.

### SUMMARY OF THE INVENTION

The present invention solves the foregoing problems. It is one object of the present invention to provide a sun hat including ear-covering portions, which provides sun protection for the front of face and ears of a wearer so as to minimize the danger of a burn through direct sunlight, by integrally extending the ear-covering portions from a peripheral portion of a visor installed at an elastic hair band of the sun hat, and by setting a visor installation angle to a relatively small acute angle.

In order to solve the above object, the present invention provides a sun hat including a circular hair band of which a rear side is open and a visor integrally formed with the hair band at a front portion thereof, characterized by comprising:

ear-covering portions rewardly extending from a peripheral portion of the visor so as to provide sun protection for a front portion and ears in a face of a wearer, wherein an installation angle of the visor installed at the hair band is set to a relatively small acute angle of about 30 to 45 degrees.

As described above, the sun hat according to the present invention comprises an elastic hair band and a visor of which ear-covering portions extend from a peripheral portion of the visor installed at the elastic hair band of the sun hat so as to provide sun protection for the front of face and ears of a wearer. Furthermore, a visor installation angle, which is defined between a tangential line of the visor and a normal line projecting from the center lower portion of the hair band, is set to a relatively small acute angle of about 30 to 45 degrees. Due to this structure, the sun hat according to the present invention provides sun protection for the front of face and ears of a wearer so as to minimize the danger of a burn through direct sunlight.

### BRIEF DESCRIPTION OF THE DRAWINGS

The above object and other characteristics and advantages of the present invention will become more apparent by describing in detail a preferred embodiment thereof with reference to the attached drawings, in which:

FIG. 1 is a perspective view of a sun hat according to a prior art;

FIG. 2 is a side view for showing the state that a user put on the sun hat according to the prior art;

FIG. 3 is a perspective view of a sun hat according to a preferred embodiment of the present invention;

FIG. 4 is a side view for showing the state that a user put on the sun hat according to the preferred embodiment of the present invention;

FIG. 5A shows a visor installation angle of the sun hat according to the prior art; and

FIG. 5B shows a visor installation angle of the sun hat according to the preferred embodiment of the present invention.

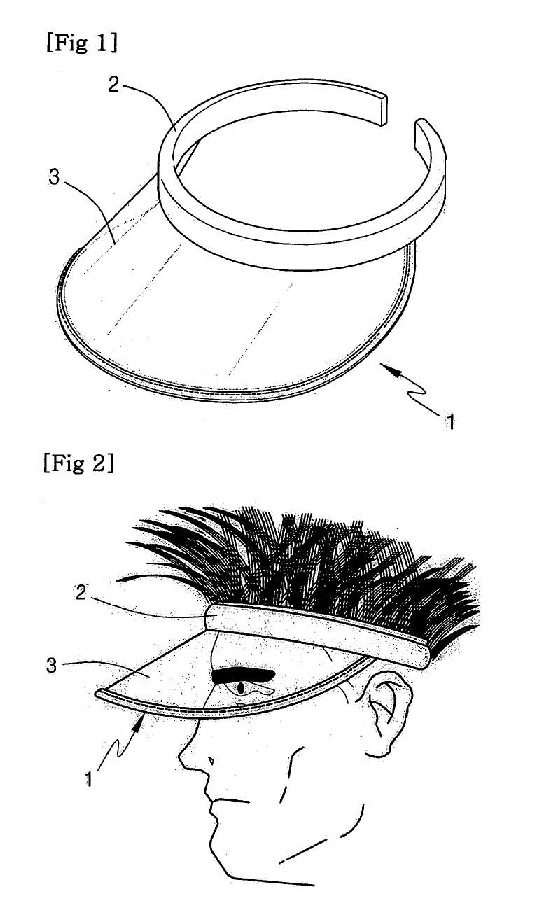

### DETAILED DESCRIPTION OF THE INVENTION

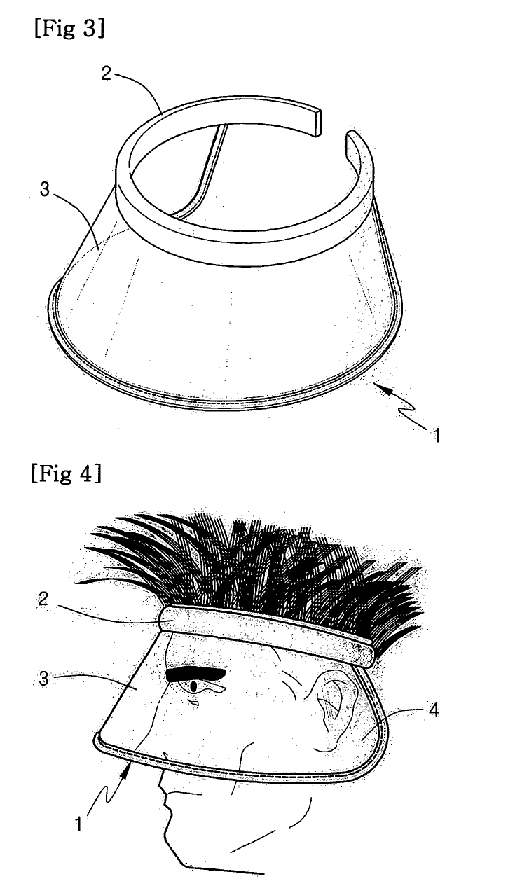

Hereinafter, the sun hat having ear-covering portions according to a preferred embodiment of the present invention will be explained in more detail with reference to the accompanying drawings FIG. 3 to FIG. 5.

The sun hat having ear-covering portions according to a preferred embodiment of the present invention comprises an elastic hair band 2 having an open rear portion and a visor 3 installed at a front side of the hair band 2. The visor 3 comprises a semitransparent body on which a UV-protection composition is coated at a predetermined thickness.

Ear-covering portions 4 extend from a peripheral portion of the visor 3 installed at the elastic hair band 2 of the sun hat 1 so as to provide sun protection for the front of face and ears of a wearer. At this time, a visor installation angle, which is defined between a tangential line of the visor and a normal line projecting from the center lower portion of the hair band, is set to a relatively small acute angle of about 30 to 45 degrees.

If the installation angle of the visor 3 installed at the elastic hair band 2 is set to a relatively small acute angle of about 30 to 45 degrees, the face of the wearer is spaced from the inner peripheral portion of the visor 3 at a distance about 3 to 5 cm. When the distance between the face of the wearer and the inner peripheral portion of the visor 3 is smaller than 3 cm, the wearer feels heavy at the face. Alternatively, if the distance between the face of the wearer and the inner peripheral portion of the visor 3 is greater than 5 cm, the quantity of the sunlight to be illuminated on the face of the wearer is getting large. Consequently, it is preferable to set the installation angle of the visor 3 at a range of 30 to 45 degrees.

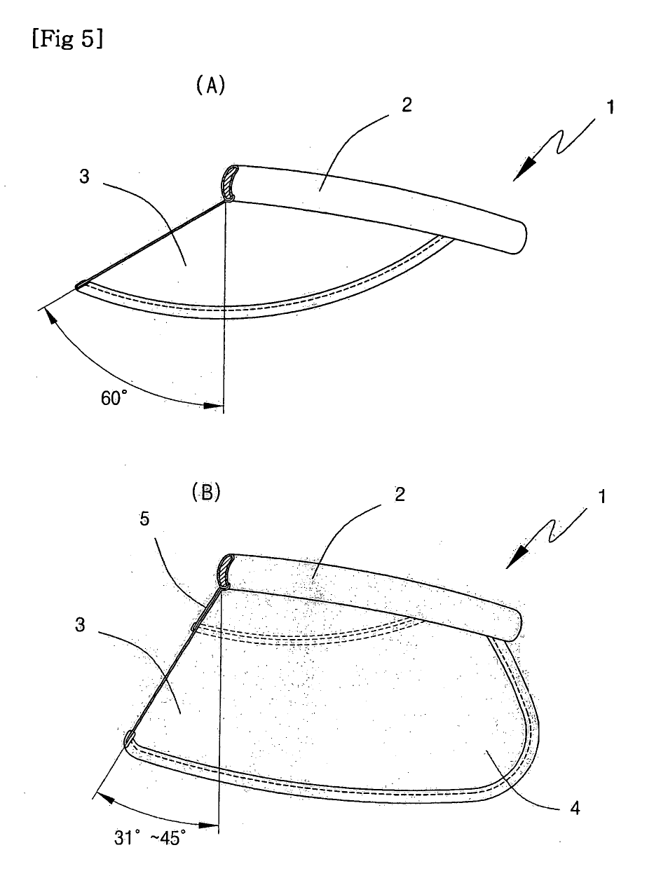

FIG. 5B shows a visor installation angle of the sun hat according to the preferred embodiment of the present invention.

As shown in FIG. 5B, a sunlight shielding part 5 is installed at an upper portion of the visor 3 so as to prevent the sunlight from illuminating wearer's eyes through the visor 3. At this time, the sunlight shielding part 5 comprises a semitransparent body on which a UV-protection composition is coated at a predetermined thickness. Alternatively, the visor 3 can be an opaque body that is made of a synthetic resin or a woven fabric.

Herein below, an operation of the present invention constituted as described above will be explained with reference to the attached drawings FIGS. 3 to 5.

When a user puts on the sun hat 1 according to the preferred embodiment of the present invention as shown in FIG. 4, ear may be shaded due to installation of the ear-covering portions 4 that are integrally formed with the visor 3 and rewardly extend from the peripheral portions of the visor 3. Thereby, ears or face portions adjacent to the ears of the wearer may be shaded so that they are not exposed to direct sunlight.

Since the installation angle of the visor 3 installed at the elastic hair band 2 is set to a relatively small acute angle of about 30 to 45 degrees, the face of the wearer is spaced from the inner peripheral portion of the visor 3 at a distance about 3 to 5 cm. Although a portion to be shaded in the face of the wearer is changed in accordance with the position of the sun around the earth, the front face portion including nose and the side face portion including ears of the wearer get always shaded from the sun. Accordingly, it is possible to screen the face of the wearer from direct sunlight.

Since the visor 3 comprises a semitransparent body on which a UV-protection composition is coated at a predetermined thickness, it is possible to prevent ultraviolet rays from being illuminated on the face of the wearer. In other words, the visor 3 made of a semitransparent body is installed at a position adjacent to eyes of the wearer, and thereby the visor 3 can have a function of a sun glace.

Referring to FIG. 5B, the sunlight shielding part 5 made of an opaque body is installed at the upper portion of the visor 3. Due to installation of the sunlight shielding part 5, an inner portion of visor 3 that is corresponding to the installation position of the sunlight shielding part 5 is shaded so that the wearer can keep his or her eyes open.

Alternatively, if the visor 3 is made of an opaque body in the same manner as that of the sun hat according to the prior art, the wearer cannot see through the visor 3. However, the visor 3 of the opaque body is not pervious to sunlight. Therefore, the front face portion and the side face portion including cheeks and ears can be shield against the sunlight. Consequently, it is possible to prevent the face of the wearer from being sunburned by the sunlight.

As described above, the sun hat according to the present invention comprises an elastic hair band and a visor of which ear-covering portions extend from a peripheral portion of the visor installed at the elastic hair band of the sun hat so as to provide sun protection for the front of face and ears of a wearer. Furthermore, a visor installation angle, which is defined between a tangential line of the visor and a normal line projecting from the center lower portion of the hair band, is set to a relatively small acute angle of about 30 to 45 degrees. When a user puts on the sun hat according to the present invention, the visor is located adjacent to the face of the wearer. Accordingly, a portion to be shaded in the face of the wearer is changed in accordance with the position of the sun around the earth, the front face portion including nose and the side face portion including ears of the wearer get always shaded from the sun. Accordingly, it is possible to screen the face of the wearer from direct sunlight so as to minimize the danger of a burn through direct sunlight.

While the present invention has been particularly shown and described with reference to the particular embodiments thereof, it will be understood by those skilled in the art that various changes in form and details may be effected therein without departing from the spirit and scope of the invention as defined by the appended claims.

1. A sun hat comprising a circular hair band of which a rear side is open and a visor integrally formed with the hair band at a front portion thereof,
ear-covering portions rewardly extending from a peripheral portion of the visor so as to provide sun protection for a front portion and ears in a face of a wearer, wherein an installation angle of the visor installed at the hair band is set to a relatively small acute angle of about 30 to 45 degrees.
